SEQUENCE LISTING (12 sequences)

SEQ ID NO: 1 - protein, 7 aa, Unknown
  source: /note=“Description of Unknown Nuclear localization signal sequence”
PKKKRKV

SEQ ID NO: 2 - protein, 7 aa, Unknown
  source: /note=“Description of Unknown Nuclear localization signal sequence”
PKKKRRV

SEQ ID NO: 3 - protein, 16 aa, Unknown
  source: /note=“Description of Unknown Nuclear localization signal sequence”
KRPAATKKAGQAKKKK

SEQ ID NO: 4 - protein, 20 aa, Unknown
  source: /note=“Description of Unknown Cell-penetrating domain sequence”
GRKKRRQRRRPPQPKKKRKV

SEQ ID NO: 5 - protein, 19 aa, Unknown
  source: /note=“Description of Unknown Cell-penetrating domain sequence”
PLSSIFSRIGDPPKKKRKV

SEQ ID NO: 6 - protein, 24 aa, Unknown
  source: /note=“Description of Unknown Cell-penetrating domain sequence”
GALFLGWLGAAGSTMGAPKKKRKV

SEQ ID NO: 7 - protein, 27 aa, Unknown
  source: /note=“Description of Unknown Cell-penetrating domain sequence”
GALFLGFLGAAGSTMGAWSQPKKKRKV

SEQ ID NO: 8 - protein, 21 aa, Unknown
  source: /note=“Description of Unknown Cell-penetrating domain sequence”
KETWWETWWTEWSQPKKKRKV

SEQ ID NO: 9 - protein, 4 aa, Artificial Sequence
  source: /note=“Description of Artificial Sequence Synthetic peptide”
DEAD

SEQ ID NO: 10 - protein, 4 aa, Artificial Sequence
  source: /note=“Description of Artificial Sequence Synthetic peptide”
DEAH

SEQ ID NO: 11 - protein, 6 aa, Artificial Sequence
  source: /note=“Description of Artificial Sequence Synthetic 6xHis tag”
HHHHHH

SEQ ID NO: 12 - protein, 9 aa, Unknown
  source: /note=“Description of Unknown LAGLIDADG family peptide motif sequence”
LAGLIDADG

(19) United States
(12) Patent Application Publication
(10) Pub. No.: US 2021/0246472 A1
(43) Pub. Date: Aug. 12, 2021

(54) DOWN-REGULATION OF THE CYTOSOLIC DNA SENSOR PATHWAY

(71) Applicant: Sigma-Aldrich Co. LLC, St. Louis, MO (US)

(72) Inventors: Nathan Zenser, St. Louis, MO (US); Dmitry Malkov, Creve Coeur, MO (US); Mark A. Gerber, St. Charles, MO (US); Jill Ward, St. Louis, MO (US)

(21) Appl. No.: 17/270,188

(22) PCT Filed: Aug. 20, 2019
(86) PCT No.: PCT/US2019/047265

§ 371 Date: Feb. 22, 2021

Related U.S. Application Data
(60) Provisional application No. 62/720,726, filed on Aug. 21, 2018.

(51) Int. Cl. C12N 15/90; C12N 9/22

(52) U.S. Cl. CPC C12N 15/907; C12N 2310/20; C12N 2800/80; C12N 9/22

(57) ABSTRACT

Methods for increasing targeted genome editing by down-regulating proteins involved in cytosolic DNA sensing pathways.

### FIELD OF THE INVENTION

The present disclosure relates to methods and compositions for increasing targeted gene editing by down-regulating proteins involved in cytosolic DNA sensing.

### SEQUENCE LISTING

The instant application contains a Sequence Listing which has been submitted electronically in ASCII format and is hereby incorporated by reference in its entirety. Said ASCII copy, created on Aug. 15, 2019, is named P18-165PCT_SL.txt and is 3,665 bytes in size.

### BACKGROUND

Specific nucleases (ZFNs, TALENs, CRISPRs/Cas9) are used to facilitate efficient targeted genome editing by creating double-strand breaks (DSBs) in DNA at desired locations. DSBs stimulate the cell's natural DNA-repair processes. One of them, the homologous recombination (HR) repair pathway, enables insertion of a transgene into the targeted region. To utilize HR, a donor template is used that contains the transgene flanked by sequences homologous to the regions on either side of the cleavage site. This donor is codelivered into the cell along with the nuclease to fool the cell by presenting the donor sequence in place of the sister chromatid to repair the cut.

Delivery of the donor DNA (or any other plasmid DNAs) is problematic for many cell lines as they are hard to transfect or nucleofect. This “sensitivity to exogenous DNA” makes the targeted transgene insertion (or targeted integration) almost impossible to achieve as it requires efficient delivery of the donor DNA. The site-specific nuclease can be added to the cells in the form of mRNA or protein, thus, bypassing the DNA sensitivity issue. However, the donor sequence must be added as DNA, traditionally as double-stranded DNA. Thus, there is a need of methods to improve or enable targeted genome editing and targeted integration in these hard-to-transfect cell lines.

Cells that are resistant to targeted transgene integration also tend to be cells that exhibit sensitivity or toxicity to foreign DNA (e.g., cell death after transfection or nucleofection of plasmid DNA). Cells have cytosolic DNA sensors that detect DNA derived from viruses and bacteria and, consequently, activate inflammatory response pathways. It is possible that down-regulation of proteins involved in the DNA sensing system would permit the introduction of donor DNA and allow for targeted integration in these cells.

### SUMMARY

Among the various aspects of the present disclosure is the provision or method for increasing targeted genome editing. The method comprises introducing a targeting endonuclease or a nucleic acid encoding the targeting endonuclease and optionally a donor DNA molecule into a cell deficient in cytosolic DNA sensing, wherein the cell deficient in cytosolic DNA sensing has a higher rate of targeted genome editing than its parental cell not deficient in cytosolic DNA sensing.

Another aspect of the present disclosure encompasses a method for increasing targeted transgene integration. The method comprises performing targeted transgene integration in a cell deficient in cytosolic DNA sensing, wherein the cell deficient in cytosolic DNA sensing has a higher rate of targeted transgene integration than its parental cell not deficient in cytosolic DNA sensing.

A further aspect of the present disclosure provides a composition comprising a cell deficient in cytosolic DNA sensing and at least one double-stranded exogenous DNA molecule, wherein the cell deficient in cytosolic DNA sensing has a higher survival rate after transfection with the at least one double-stranded exogenous DNA molecule than a parental cell not deficient in cytosolic DNA sensing.

Other aspects and iterations of the disclosure are described in more detail below.

### BRIEF DESCRIPTION OF THE FIGURES

FIGS. 1A and 1B present cell sorter analyses of THP-1 wild type and THP-1 KO (MD21D1) cells nucleofected with ZFNs targeted to the actin locus and a BFP donor plasmid.

FIGS. 2A-2D show cell sorter analyses of THP-1 KO (MD21D1) cells nucleofected with ZFNs targeted to the tubulin locus and a GFP donor plasmid.

### DETAILED DESCRIPTION

The present disclosure provides methods and compositions for increasing targeted genome editing or targeted transgene integration. The method comprises down-regulating proteins involved in cytosolic DNA sensing, such that the cells are less sensitive to exogenous DNA molecules. As a consequence, targeted genome editing/integration can be performed in said cells.

### (I) Cells Deficient in Cytosolic DNA Sensing

The methods disclosed herein utilize cells in which cytosolic DNA sensing pathways have been down-regulated such that the cells are less sensitive to exogenous DNA (e.g., donor DNA or plasmid DNA). As a consequence, the efficiency of targeted genome editing can be improved in said cells deficient in cytosolic DNA sensing.

In general, the cells deficient in cytosolic DNA sensing are engineered to be deficient in one or more proteins involved in cytosolic DNA sensing pathways. Thus, the engineered cells lack or have reduced levels of the one or more proteins involved in cytosolic DNA sensing as compared to the non-engineered parent cells. Expression of the one or more proteins involved in cytosolic DNA sensing can be transiently knocked-down by RNA interference (RNAi) or CRISPR interference (CRISPRi). Alternatively, expression of the one or more proteins involved in cytosolic DNA sensing can be permanently knocked-out by targeted gene inactivation with targeting endonucleases. As a consequence of the deficiency in the cytosolic DNA sensing pathway, the engineered cell lines are less sensitive to exogenous DNA and have higher rates of targeted genome editing than their non-engineered parental cell lines.

(a) Proteins Involved in Cytosolic DNA Sensing Pathways

The innate immune system comprises cytosolic DNA sensing pathways that detect cytosolic double-stranded DNA (dsDNA), which is indicative of cellular damage and/or bacterial or viral infection. Cytosolic dsDNA is recognized by DNA sensors that activate signaling pathways that trigger the activation of inflammatory genes, thereby resulting in anti-viral protection, anti-bacterial protection, natural killer cell activation, or cell death.

Sensors of cytosolic dsDNA include cyclic GMP-AMP synthase (cGAS; gene symbol is MB21D1), interferon gamma inducible protein 16 (IFI16; same gene symbol), DEAD-box helicase 41 (DDX41; same gene symbol), leucine rich repeat (in flightless I) interacting protein (LRRFIP1; same gene symbol), DNA-dependent activator of interferon (IFN)-regulatory factors (DAI; gene symbol is ZBP1), DEAH-box helicase 8 (DHX9, same gene symbol), DEAH-box helicase (DHX36; same gene symbol), absent in melanoma 2 (AIM2; same gene symbol), X-ray repair cross-complementing protein 6 (Ku70; gene symbol is XRCC6) and RNA polymerase III (Pol III; gene symbol is POLR3A). A downstream protein activated by some cytosolic DNA sensors (e.g., cGAS) is stimulator of interferon genes (STING; gene symbol is TMEM173). In some embodiments, the cell is deficient in cGAS. In other embodiments, the cell is deficient in STING. In still other embodiments, the cell is deficient in both cGAS and STING.

(b) Permanent Deficiency in Cytosolic DNA Sensing

In some embodiments, the cell deficient in cytosolic DNA sensing can have a permanent deficiency. For example, the cell deficient in cytosolic DNA sensing comprises at least one inactivated chromosomal sequence encoding the protein involved in cytosolic DNA sensing. The chromosomal sequences encoding the protein involved in cytosolic DNA sensing can be inactivated with a targeting endonuclease-mediated genome editing technique, which is described below in section (II).

A targeting endonuclease comprises a DNA-binding domain and a nuclease domain. The DNA-binding domain of the targeting endonuclease is programmable, meaning that it can be designed or engineered to recognize and bind different DNA sequences. In some embodiments, the DNA binding is mediated by interactions between the DNA-binding domain of the targeting endonuclease and the target DNA. Thus, the DNA-binding domain can be programmed to bind a DNA sequence of interest by protein engineering. In other embodiments, DNA binding is mediated by a guide RNA that interacts with the DNA-binding domain of the targeting endonuclease and the target DNA. In such instances, the DNA-binding domain can be targeted to a DNA sequence of interest by designing the appropriate guide RNA.

Suitable targeting endonuclease include zinc finger nucleases, clustered regularly interspersed short palindromic repeats (CRISPR)/CRISPR-associated (Cas) (CRISPR/Cas) nuclease systems, CRISPR/Cas dual nickase systems, transcription activator-like effector nucleases, meganucleases, or fusion proteins comprising programmable DNA-binding domains and nuclease domains. The targeting endonuclease can comprise wild-type or naturally-occurring DNA-binding and/or nuclease domains, modified versions of naturally-occurring DNA-binding and/or nuclease domains, synthetic or artificial DNA-binding and/or nuclease domains, or combinations thereof.

(i) Zinc Finger Nucleases

In some embodiments, the targeting endonuclease can be a zinc finger nuclease (ZFN). A ZFN comprises a DNA-binding zinc finger region and a nuclease domain. The zinc finger region can comprise from about two to seven zinc fingers, for example, about four to six zinc fingers, wherein each zinc finger binds three nucleotides, and wherein the zinc fingers can be linked together using suitable linker sequences. The zinc finger region can be engineered to recognize and bind to any DNA sequence. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. Publically available web-based tools for identifying potential target sites in DNA sequences as well as designing zinc finger binding domains are known in the art.

A ZFN also comprises a nuclease domain, which can be obtained from any endonuclease or exonuclease. Non-limiting examples of endonucleases from which a nuclease domain can be derived include, but are not limited to, restriction endonucleases and homing endonucleases. A cleavage domain also may be derived from an enzyme or portion thereof that requires dimerization for cleavage activity. Two zinc finger nucleases may be required for cleavage, as each nuclease comprises a monomer of the active enzyme dimer. When two cleavage monomers are used to form an active enzyme dimer, the recognition sites for the two zinc finger nucleases are generally disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides.

In some embodiments, the nuclease domain can be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. In some embodiments, the nuclease domain can be a FokI nuclease domain or a derivative thereof. The type II-S nuclease domain can be modified to facilitate dimerization of two different nuclease domains. For example, the cleavage domain of FokI can be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. For example, one modified FokI domain can comprise Q486E, I499L, and/or N496D mutations, and the other modified FokI domain can comprise E490K, I538K, and/or H537R mutations.

The ZFN can further comprise at least one nuclear localization signal, cell-penetrating domain, and/or marker domain. Non-limiting examples of nuclear localization signals include PKKKRKV (SEQ ID NO:1), PKKKRRV (SEQ ID NO:2), or KRPAATKKAGQAKKKK (SEQ ID NO:3). Examples of suitable cell-penetrating domains include, without limit, GRKKRRQRRRPPQPKKKRKV (SEQ ID NO:4), PLSSIFSRIGDPPKKKRKV (SEQ ID NO:5), GALFLGWLGAAGSTMGAPKKKRKV (SEQ ID NO:6), GALFLGFLGAAGSTMGAWSQPKKKRKV (SEQ ID NO: 7), or KETVVWETVWVTEWSQPKKKRKV (SEQ ID NO: 8). Non-limiting examples of marker domains include fluorescent proteins (e.g., GFP, eGFP, RFP, BFP, and the like), and purification or epitope tags (e.g., 6× His, FLAG, HA, GST, and so forth). The nuclear localization signal, cell-penetrating domain, and/or marker domain can be located at the N-terminus, the C-terminal, or in an internal location of the protein.

(ii) CRISPR/Cas Nuclease Systems

In other embodiments, the targeting endonuclease can be a RNA-guided CRISPR/Cas nuclease system, which introduces a double-stranded break in the DNA. The CRISPR/Cas nuclease system comprises a CRISPR/Cas nuclease and a guide RNA.

The CRISPR/Cas nuclease can be derived from a type I (i.e., IA, IB, IC, ID, IE, or IF), type II (i.e., IIA, IIB, or IIC), type III (i.e., IIIA or IIIB), or type V CRISPR system, which are present in various bacteria and archaea. The CRISPR/Cas system can be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR nuclease include Cas proteins, Cpf proteins, Cmr proteins, Csa proteins, Csb proteins, Csc proteins, Cse proteins, Csf proteins, Csm proteins, Csn proteins, Csx proteins, Csy proteins, Csz proteins, and derivatives or variants thereof. In specific embodiments, the CRISPR/Cas nuclease can be a type II Cas9 protein, a type V Cpf1 protein, or a derivative thereof. In some embodiments, the CRISPR/Cas nuclease can be Streptococcus pyogenes Cas9 (SpCas9) or Streptococcus thermophilus Cas9 (StCas9). In other embodiments, the CRISPR/Cas nuclease can be Campylobacter jejuni Cas9 (CjCas9). In alternate embodiments, the CRISPR/Cas nuclease can be Francisella novicida Cas9 (FnCas9). In yet other embodiments, the CRISPR/Cas nuclease can be Francisella novicida Cpf1 (FnCpf1).

In general, the CRISPR/Cas nuclease comprises a RNA recognition and/or RNA binding domain, which interacts with the guide RNA. The CRISPR/Cas nuclease also comprises at least one nuclease domain having endonuclease activity. For example, a Cas9 protein can comprise a RuvC-like nuclease domain and a HNH-like nuclease domain, and a Cpf1 protein can comprise a RuvC-like domain. CRISPR/Cas nucleases can also comprise DNA binding domains, helicase domains, RNase domains, protein-protein interaction domains, dimerization domains, as well as other domains.

The CRISPR/Cas nuclease can further comprise at least one nuclear localization signal, cell-penetrating domain, marker domain, and/or detectable label, which are described above in section (I)(b)(i).

The CRISPR/Cas nuclease system also comprises a guide RNA (gRNA). The guide RNA interacts with the CRISPR/Cas nuclease to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY and PAM sequences for Cpf1 include 5′-TTN (wherein N is defined as any nucleotide, W is defined as either A or T, and Y is defined an either C or T). Each gRNA comprises a sequence that is complementary to the target sequence (e.g., a Cas9 gRNA can comprise GN₁₇₋₂₀GG). The gRNA can also comprise a scaffold sequence that forms a stem loop structure and a single-stranded region. The scaffold region can be the same in every gRNA. In some embodiments, the gRNA can be a single molecule (i.e., sgRNA). In other embodiments, the gRNA can be two separate molecules.

(iii) CRISPR/Cas Nickase Systems

In other embodiments, the targeting endonuclease can be a CRISPR/Cas nickase system. CRISPR/Cas nickase systems are similar to the CRISPR/Cas nuclease systems described above except that the CRISPR/Cas nuclease is modified to cleave only one strand of DNA. Thus, a single CRISPR/Cas nickase system creates a single-stranded break or nick in the DNA. Alternatively, a paired CRISPR/Cas nickase system (or dual nickase system) comprising a pair of offset gRNAs can create a double-stranded break in the DNA by generating closely spaced single-stranded breaks on opposite strands of the DNA.

A CRISPR/Cas nuclease can be converted to a nickase by one or more mutations and/or deletions. For example, a Cas9 nickase can comprise one or more mutations in one of the nuclease domains, wherein the one or more mutations can be D10A, E762A, and/or D986A in the RuvC-like domain or the one or more mutations can be H840A, N854A and/or N863A in the HNH-like domain.

(iv) Transcription Activator-Like Effector Nucleases

In alternate embodiments, the targeting endonuclease can be a transcription activator-like effector nuclease (TALEN). TALENs comprise a DNA-binding domain composed of highly conserved repeats derived from transcription activator-like effectors (TALEs) that is linked to a nuclease domain. TALEs are proteins secreted by plant pathogen Xanthomonas to alter transcription of genes in host plant cells. TALE repeat arrays can be engineered via modular protein design to target any DNA sequence of interest. The nuclease domain of TALENs can be any nuclease domain as described above in section (I)(b)(i). In specific embodiments, the nuclease domain is derived from FokI (Sanjana et al., 2012, Nat Protoc, 7(1):171-192).

The TALEN can also comprise at least one nuclear localization signal, cell-penetrating domain, marker domain, and/or detectable label, which are described above in section (I)(b)(i).

(v) Meganucleases or Rare-Cutting Endonucleases

In still other embodiments, the targeting endonuclease can be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. In some embodiments, the meganuclease can be I-SceI, I-TevI, or variants thereof. A meganuclease can be targeted to a specific chromosomal sequence by modifying its recognition sequence using techniques well known to those skilled in the art.

In alternate embodiments, the targeting endonuclease can be a rare-cutting endonuclease or derivative thereof. Rare-cutting endonucleases are site-specific endonucleases whose recognition sequence occurs rarely in a genome, preferably only once in a genome. The rare-cutting endonuclease may recognize a 7-nucleotide sequence, an 8-nucleotide sequence, or longer recognition sequence. Non-limiting examples of rare-cutting endonucleases include NotI, AscI, PacI, AsiSI, SbfI, and FseI.

The meganuclease or rare-cutting endonuclease can also comprise at least one nuclear localization signal, cell-penetrating domain, marker domain, and/or detectable label, which are described above in section (I)(b)(i).

(vi) Fusion Proteins Comprising Nuclease Domains

In yet additional embodiments, the targeting endonuclease can be a fusion protein comprising a nuclease domain and a programmable DNA-binding domain. The nuclease domain can be any of those described above in section (I)(b)(i), a nuclease domain derived from a CRISPR/Cas nuclease (e.g., RuvC-like or HNH-like nuclease domains of Cas9 or nuclease domain of Cpf1), or a nuclease domain derived from a meganuclease or rare-cutting endonuclease.

The programmable DNA-binding domain of the fusion protein can be derived from a targeting endonuclease (i.e., CRISPR/CAS nuclease or meganuclease) that is modified to lack all nuclease activity (i.e., is catalytically inactive). Alternatively, the programmable DNA-binding domain of the fusion protein can be a programmable DNA-binding protein such as, e.g., a zinc finger protein or a TALE.

In some embodiments, the programmable DNA-binding domain can be a catalytically inactive CRISPR/Cas nuclease in which the nuclease activity was eliminated by mutation and/or deletion. For example, the catalytically inactive CRISPR/Cas protein can be a catalytically inactive (dead) Cas9 (dCas9) in which the RuvC-like domain comprises a D10A, E762A, and/or D986A mutation and the HNH-like domain comprises a H840A, N854A and/or N863A mutation. Alternatively, the catalytically inactive CRISPR/Cas protein can be a catalytically inactive (dead) Cpf1 protein comprising comparable mutations in the nuclease domain. In other embodiments, the programmable DNA-binding domain can be a catalytically inactive meganuclease in which nuclease activity was eliminated by mutation and/or deletion, e.g., the catalytically inactive meganuclease can comprise a C-terminal truncation.

The fusion protein comprising a nuclease domain can also comprise at least one nuclear localization signal, cell-penetrating domain, marker domain, and/or detectable label, which are described above in section (I)(b)(i).

(c) Transient Deficiency in Cytosolic DNA Sensing

In other embodiments, the cell deficient in cytosolic DNA sensing can have a transient deficiency. For example, expression of the protein involved in cytosolic DNA sensing can be transiently knocked-down by RNA interference (RNAi) or CRISPR interference (CRISPRi). As such the cell deficient in cytosolic DNA sensing can comprise a RNAi agent targeted to a transcript (mRNA) of the protein involved in cytosolic DNA sensing or a CRISPRi agent targeted to a chromosomal DNA sequence encoding the protein involved in cytosolic DNA sensing.

(i) RNAi

RNA interference refers to a process by which an RNAi agent inhibits expression of a target transcript by cleavage of the transcript or by disrupting translation of the transcript into protein. RNAi agents include short interfering RNA (siRNA) and short hairpin RNA (shRNA). In general, a siRNA comprises a double-stranded RNA molecule that ranges from about 15 to about 29 nucleotides in length, or more generally from about 19 to about 23 nucleotides in length. In specific embodiments, the siRNA can be about 21 nucleotides in length. The siRNA can optionally further comprise one or two single-stranded overhangs, e.g., a 3′ overhang on one or both ends. The siRNA can be formed from two RNA molecules that hybridize together or, alternatively, can be generated from a short hairpin RNA (shRNA) (see below). In some embodiments, the two strands of the siRNA can be completely complementary, such that no mismatches or bulges exist in the duplex formed between the two sequences. In other embodiments, the two strands of the siRNA can be substantially complementary, such that one or more mismatches and/or bulges exist in the duplex formed between the two sequences. In certain embodiments, one or both of the 5′ ends of the siRNA can have a phosphate group, while in other embodiments one or both of the 5′ ends can lack a phosphate group.

One strand of the siRNA, which is referred to as the “antisense strand” or “guide strand,” includes a portion that hybridizes with the target transcript. In some embodiments, the antisense strand of the siRNA can be completely complementary to a region of the target transcript, i.e., it hybridizes to the target transcript without a single mismatch or bulge throughout the length of the siRNA. In other embodiments, the antisense strand can be substantially complementary to the target region, i.e., one or more mismatches and/or bulges can exist in the duplex formed by the antisense strand and the target transcript. Typically, siRNAs are targeted to exonic sequences of the target transcript. Those of skill in the art are familiar with programs, algorithms, and/or commercial services that design siRNAs for target transcripts.

In general, a shRNA is an RNA molecule comprising at least two complementary portions that are hybridized or are capable of hybridizing to form a double-stranded structure sufficiently long to mediate RNA interference, and at least one single-stranded portion that forms a loop connecting the regions of the shRNA that form the duplex. The structure can also be called a stem-loop structure, with the stem being the duplex portion. In some embodiments, the duplex portion of the structure can be completely complementary, such that no mismatches or bulges exist in the duplex region of the shRNA. In other embodiments, the duplex portion of the structure can be substantially complementary, such that one or more mismatches and/or bulges can exist in the duplex portion of the shRNA. The loop of the structure can be from about 1 to about 20 nucleotides in length, specifically from about 6 to about 9 nucleotides in length. The loop can be located at either the 5′ or 3′ end of the region that is complementary to the target transcript (i.e., the antisense portion of the shRNA).

The shRNA can further comprise an overhang on the 5′ or 3′ end. The optional overhang can be from about 1 to about 20 nucleotides in length, or more specifically from about 2 to about 15 nucleotides in length. In some embodiments, the overhang can comprise one or more U residues, e.g., between about 1 and about 5 U residues. In some embodiments, the 5′ end of the shRNA can have a phosphate group. In general, shRNAs are processed into siRNAs by the conserved cellular RNAi machinery. Thus, shRNAs are precursors of siRNAs and are similarly capable of inhibiting expression of a target transcript that is complementary of a portion of the shRNA (i.e., the antisense portion of the shRNA). Those of skill in the art are familiar with the available resources for the design and synthesis of shRNAs. An exemplary example is MISSION® shRNAs (Sigma-Aldrich).

The siRNA or shRNA can be introduced into the cell as RNA. Alternatively, the siRNA or shRNA can be expressed in vivo from an RNAi expression construct. Suitable constructs include plasmid vectors, phagemids, cosmids, artificial/mini-chromosomes, transposons, and viral vectors (e.g., lentiviral vectors, adeno-associated viral vectors, etc.). In one embodiment, the RNAi expression construct can be a plasmid vector (e.g., pUC, pBR322, pET, pBluescript, and variants thereof). The RNAi expression construct can comprise two promoter control sequences, wherein each is operably linked appropriate coding sequence such that two separate, complementary siRNA strands can be transcribed. The two promoter control sequences can be in the same orientation or in opposite orientations. In another embodiment, the RNAi expression vector can contain a promoter control sequence that drives transcription of a single RNA molecule comprising two complementary regions, such that the transcript forms a shRNA. In general, the promoter control sequence(s) will be RNA polymerase III (Pol III) promoters such as U6 or H1 promoters. In other embodiments, RNA polymerase II (Pol II) promoter control sequences can be used. The RNAi expression constructs can contain additional sequence elements, such as transcription termination sequences, selectable marker sequences, etc. The RNAi expression construct can be introduced into the cell line of interest using standard procedures. The RNAi agent or RNAi expression vector can be introduced into the cell using methods well known to those of skill in the art (see, e.g., section (II)(b) below).

(ii) CRISPRi

CRISPR interference refers to a process in which gene expression is inhibited by the binding of a catalytically inactive CRISPR/Cas system (CRISPRi) to a target site in genomic DNA. The CRISPRi agent comprises a catalytically inactive CRISPR/Cas nuclease in which all nuclease activity was eliminated by mutation and/or deletion. For example, the catalytically inactive CRISPR/Cas protein can be a catalytically inactive (dead) Cas9 (dCas9) in which the RuvC-like domain comprises a D10A, E762A, and/or D986A mutation and the HNH-like domain comprises a H840A, N854A and/or N863A mutation. In some embodiments, the catalytically inactive (dead) CRISPR/Cas protein can be fused with a transcriptional repressor domain. Suitable transcriptional repressor domains include Kruppel-associated box A (KRAB-A) repressor domains, inducible cAMP early repressor (ICER) domains, YY1 glycine rich repressor domains, Sp1-like repressors, E(spl) repressors, IκB repressor, and MeCP2.

The guide RNA of the CRISPR/Cas system targets the CRISPR/Cas system to a targeted site in the chromosomal sequence encoding the protein involved in cytosolic DNA sensing. For example, the guide RNA can target the catalytically inactive CRISPR/Cas system to a target site in the promoter region such that transcription initiation is repressed. Alternatively, the guide RNA can target the catalytically inactive CRISPR/Cas system to a target site in the transcribed region of the gene such that transcription elongation is repressed.

The CRISPRi agent can be introduced into the cell as a protein-RNA complex. In other embodiments, a nucleic acid (i.e., RNA or DNA) encoding the catalytically inactive CRISPR/Cas protein can be introduced into the cell along with the guide RNA. In still other embodiment, a DNA expression construct encoding the catalytically inactive CRISPR/Cas protein and the guide RNA can be introduced into the cell. Those skilled in the art are familiar with means for introducing the CRISPRi agent or nucleic acid encoding the CRISPRi agent (see, e.g., section (II)(b) below).

(d) Cell Types

In general, the cell deficient in cytosolic DNA sensing is a mammalian cell or, more specifically, a mammalian cell line. In some embodiments, the mammalian cell line is a hematopoietic cell line. Non-limiting examples of suitable hematopoietic cell lines include THP-1 (human monocytic cells), HL-60 (human promyelocytic leukemia cells), U-937 (human macrophage cells), Ramos (human Burkitt's lymphoma cells), Jurkat (human T lymphocyte cells), Daudi (human B lymphoblast cells), IM-9 (human B lymphoblast cells), ARH-77 (human B lymphoblast cells), RPMI 8226 (human B lymphocyte cells), MC/CAR (human B lymphocyte cells), RPMI 1788 (human B lymphocytes), TF-1 (human erythroblast cells), K562 (human myelogenous leukemia cells), RAW 264.7 (mouse macrophage cells), RBL (rat B lymphoma cells), DH82 (rat monocyte/macrophage cells), NS0 (mouse myeloma cells), or SP2/0 (mouse myeloma cells). In additional embodiments, the cell line can be a hematopoietic stem cell line.

In other embodiments, the mammalian cell line can be HEK293 or HEK293T (human embryonic kidney cells), HELA (human cervical carcinoma cells), W138 (human lung cells), Hep G2 (human liver cells), U2-OS (human osteosarcoma cells), A549 (human alveolar basal epithelial cells), A-431 (human carcinoma cells), COS7 (monkey kidney SV-40 transformed fibroblasts), CVI-76 (monkey kidney cells), VERO-76 (African green monkey kidney cells), CMT (canine mammary cells), MDCK (canine kidney cells), 9L (rat glioblastoma cells), B35 (rat neuroblastoma cells), HTC (rat hepatoma cells), BRL 3A (buffalo rat liver cells), D17 (rat osteosarcoma cells), CHO (Chinese hamster ovary cells), BHK (baby hamster kidney cells), NIH3T3 (mouse embryonic fibroblasts), A20 (mouse B lymphoma cells), B16 (mouse melanoma cells), C2C12 (mouse myoblast cells), C3H-10T1/2 (mouse embryonic mesenchymal cells), CT26 (mouse carcinoma cells), DuCuP (mouse prostate cells), EMT6 (mouse breast cells), Nepal c1c7 (mouse hepatoma cells), J5582 (mouse myeloma cells), MTD-1A (mouse epithelial cells), MyEnd (mouse myocardial cells), RenCa (mouse renal cells), RIN-5F (mouse pancreatic cells), X64 (mouse melanoma cells), YAC-1 (mouse lymphoma cells).

In specific embodiments, the cell line is a THP-1, HL-60, U-937, Ramos, or Jurkat cell line.

(e) Properties of the Cells Deficient in Cytosolic DNA Sensing

Cells deficient in cytosolic DNA sensing are less sensitive to the toxic effects of exogenous DNA as compared to parental cells that are not deficient in cytosolic DNA sensing. In some embodiments, cells deficient in cytosolic DNA sensing have higher survival rates after transfection with at least one double-stranded DNA molecules than parental cells not deficient in cytosolic DNA sensing. The rate of survival can be increased by at least about 25%, at least about 50%, at least about 100%, at least about 200%, at least about 400%, or more than about 400%.

In other embodiments, cells deficient in cytosolic DNA sensing have higher rates of targeted genome editing or targeted transgene integration than parental cells not deficient in cytosolic DNA sensing. The rate of genome editing or targeted integration can be increased by at least about 25%, at least about 50%, at least about 100%, at least about 200%, at least about 400%, or more than about 400%.

The cells deficient in cytosolic DNA sensing have comparable growth rates as parental cells not deficient in cytosolic DNA sensing.

### (II) Methods for Increasing the Efficiency of Targeted Genome Editing

A further aspect of the present disclosure encompasses method for increasing the efficiency of targeted genome editing (e.g., transgene integration) by performing the targeted genome editing in cells that are deficient in cytosolic DNA sensing. Because cells deficient in cytosolic DNA sensing are less sensitive to exogenous DNA, said cells can have higher rates of targeted genome editing than parental cells not deficient in cytosolic DNA sensing.

(a) Reagents for the Method

The method comprises introducing a targeting endonuclease or nucleic acid encoding the targeting endonuclease and optionally a donor DNA molecule into cells deficient in cytosolic DNA sensing. The cells deficient in cytosolic DNA sensing are described above in section (I). The targeting endonuclease can be a ZFN, a CRISPR/Cas nuclease system, a CRISPR/Cas dual nickase system, a TALEN, a meganucleases, or a fusion protein comprising a programmable DNA-binding domain and a nuclease domain, which are detailed above in section (I)(b). Nucleic acids encoding the targeting endonuclease and the optional donor DNA molecule are detailed below.

(i) Nucleic Acids Encoding Targeting Endonucleases

The nucleic acid encoding the targeting endonuclease protein can be DNA or RNA, linear or circular, single-stranded or double-stranded. The RNA or DNA can be codon optimized for efficient translation into protein in the mammalian cell of interest. Codon optimization programs are available as freeware or from commercial sources. In some embodiments, the nucleic acid encoding the targeting endonuclease can be mRNA. The mRNA can be 5′ capped and/or 3′ polyadenylated. In other embodiments, the nucleic acid encoding the targeting endonuclease can be DNA. In general, the DNA encoding the target endonuclease is double-stranded DNA. The coding DNA can be operably linked to promoter control sequences, polyadenylation signals (e.g., SV40 polyA signal, bovine growth hormone (BGH) polyA signal, etc.), and/or transcriptional termination sequences.

In some embodiments, the DNA sequence encoding the targeting endonuclease can be operably linked to a promoter control sequence for expression in the mammalian cell of interest. The promoter control sequence can be constitutive, regulated, or cell- or tissue-specific. Suitable constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable regulated promoter control sequences include without limit those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter. The promoter control sequence can be wild type or it can be modified for more efficient or efficacious expression.

In other embodiments, the DNA sequence encoding the targeting endonuclease can be operably linked to a promoter sequence that is recognized by a phage RNA polymerase for in vitro mRNA synthesis. In such embodiments, the in vitro-transcribed RNA can be purified, capped, and/or polyadenylated and introduced into the cell of interest. For example, the promoter sequence can be a T7, T3, or SP6 promoter sequence or a variation of a T7, T3, or SP6 promoter sequence.

In still other embodiments, the DNA sequence encoding the targeting endonuclease can be operably linked to a promoter sequence for in vitro expression in bacterial or eukaryotic cells. Suitable bacterial promoters include, without limit, T7 promoters, lac operon promoters, trp promoters, tac promoters (which are hybrids of trp and lac promoters), variations thereof any of the foregoing, and combinations thereof of any of the foregoing. Non-limiting examples of suitable eukaryotic promoters are listed above. In such embodiments, the expressed protein can be purified for introduction into the cell of interest.

In various embodiments, the DNA encoding the targeting endonuclease can be present in a DNA construct. Suitable constructs include plasmid vectors, phagemids, cosmids, artificial/mini-chromosomes, transposons, and viral vectors (e.g., lentiviral vectors, adeno-associated viral vectors, etc.). In one embodiment, the DNA encoding the targeting endonuclease is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

In embodiments in which the targeting endonuclease comprises a CRISPR/Cas protein or variant thereof, the expression vector comprising the DNA sequence encoding the CRISPR/Cas protein or variant thereof can further comprise DNA sequence encoding one or more guide RNAs. The sequence encoding the guide RNA(s) generally is operably linked to at least one transcriptional control sequence for expression of the guide RNA(s) in the cell of interest. For example, DNA encoding the guide RNA(s) can be operably linked to a promoter sequence that is recognized by RNA polymerase III (Pol III). Examples of suitable Pol III promoters include, but are not limited to, mammalian U6, U3, H1, and 7SL RNA promoters.

(ii) Optional Donor DNA Molecule

In some embodiments, the method further comprises introducing a donor DNA molecule into the cell deficient in cytosolic DNA sensing. In general, the donor DNA molecule is double-stranded DNA. The donor DNA molecule can be linear or circular. In some embodiments, the donor DNA molecule can be a vector, e.g., a plasmid vector.

The donor DNA molecules comprises at least one donor sequence. In some aspects, the donor sequence of the donor DNA molecules can be a modified version of an endogenous or native chromosomal sequence. For example, the donor sequence can be essentially identical to a portion of the chromosomal sequence at or near the sequence targeted by the targeting endonuclease, but which comprises at least one nucleotide change. Thus, upon integration or exchange with the native sequence, the sequence at the targeted chromosomal location comprises at least one nucleotide change. For example, the change can be an insertion of one or more nucleotides, a deletion of one or more nucleotides, a substitution of one or more nucleotides, or combinations thereof. As a consequence of the integration of the modified sequence, the cell can produce a modified gene product from the targeted chromosomal sequence.

In other aspects, the donor sequence of the donor DNA molecules can be an exogenous sequence. As used herein, an “exogenous” sequence refers to a sequence that is not native to the cell, or a sequence whose native location is in a different location in the genome of the cell. For example, the exogenous sequence can comprise protein coding sequence, which can be operably linked to an exogenous promoter control sequence such that, upon integration into the genome, the cell is able to express the protein coded by the integrated sequence (i.e., a transgene). Alternatively, the exogenous sequence can be integrated into the chromosomal sequence such that its expression is regulated by an endogenous promoter control sequence. In other iterations, the exogenous sequence can be a transcriptional control sequence, another expression control sequence, an RNA coding sequence, and so forth. Integration of an exogenous sequence into a chromosomal sequence is termed a “knock-in.”

As can be appreciated by those skilled in the art, the length of the donor sequence can and will vary. For example, the donor sequence can vary in length from several nucleotides to hundreds of nucleotides to hundreds of thousands of nucleotides.

Typically, the donor sequence in the donor DNA molecule polynucleotide is flanked by at least one sequence having substantial sequence identity with a sequence at or near the site that is targeted by the targeting endonuclease. For example the donor sequence can be flanked by an upstream sequence and a downstream sequence, which have substantial sequence identity to sequences located upstream and downstream, respectively, of the sequence targeted by the targeting endonuclease. Because of these sequence similarities, the upstream and downstream sequences of the donor DNA molecule permit homologous recombination between the donor DNA molecule and the targeted chromosomal sequence such that the donor sequence can be integrated into (or exchanged with) the chromosomal sequence.

The upstream sequence, as used herein, refers to a nucleic acid sequence that shares substantial sequence identity with a chromosomal sequence upstream of the sequence targeted by the targeting endonuclease. Similarly, the downstream sequence refers to a nucleic acid sequence that shares substantial sequence identity with a chromosomal sequence downstream of the sequence targeted by the targeting endonuclease. As used herein, the phrase “substantial sequence identity” refers to sequences having at least about 75% sequence identity. Thus, the upstream and downstream sequences in the donor polynucleotide can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% A sequence identity with sequence upstream or downstream to the target sequence. In an exemplary embodiment, the upstream and downstream sequences in the donor DNA molecule can have about 95% or 100% sequence identity with chromosomal sequences upstream or downstream to the sequence targeted by the targeting endonuclease.

In some embodiments, the upstream sequence shares substantial sequence identity with a chromosomal sequence located immediately upstream of the sequence targeted by the targeting endonuclease. In other embodiments, the upstream sequence shares substantial sequence identity with a chromosomal sequence that is located within about one hundred (100) nucleotides upstream from the target sequence. Thus, for example, the upstream sequence can share substantial sequence identity with a chromosomal sequence that is located about 1 to about 20, about 21 to about 40, about 41 to about 60, about 61 to about 80, or about 81 to about 100 nucleotides upstream from the target sequence. In some embodiments, the downstream sequence shares substantial sequence identity with a chromosomal sequence located immediately downstream of the sequence targeted by the targeting endonuclease. In other embodiments, the downstream sequence shares substantial sequence identity with a chromosomal sequence that is located within about one hundred (100) nucleotides downstream from the target sequence. Thus, for example, the downstream sequence can share substantial sequence identity with a chromosomal sequence that is located about 1 to about 20, about 21 to about 40, about 41 to about 60, about 61 to about 80, or about 81 to about 100 nucleotides downstream from the target sequence.

Each upstream or downstream sequence can range in length from about 20 nucleotides to about 5000 nucleotides. In some embodiments, upstream and downstream sequences can comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2800, 3000, 3200, 3400, 3600, 3800, 4000, 4200, 4400, 4600, 4800, or 5000 nucleotides. In specific embodiments, upstream and downstream sequences can range in length from about 50 to about 1500 nucleotides.

(b) Delivery to the Cell

The method comprises introducing the targeting endonuclease or nucleic acid encoding the targeting endonuclease and the optional donor DNA molecule into the cell of interest. In some embodiments, the targeting endonuclease can be delivered to and introduced into the cell as a protein or as a protein-nucleic acid complex (i.e., in embodiments in which the targeting endonuclease is a RNA-guided CRISPR/Cas system). In other embodiments, the targeting endonuclease can be delivered to and introduced into the cell as a mRNA. In still other embodiments, the targeting endonuclease can be delivered to and introduced into the cell as DNA, e.g., as part of an expression construct. As mentioned above, in embodiments in which the targeting endonuclease is a CRISPR/Cas system, the expression construct can also contain sequence encoding the guide RNA comprises a

The targeting endonuclease molecule(s) and the optional donor DNA molecule can be introduced into the cell by a variety of means. Suitable delivery means include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In specific embodiments, the targeting endonuclease molecule(s) and the optional donor DNA molecule can be introduced into the cell by nucleofection.

In embodiments in which more than one targeting endonuclease molecule and more than one donor DNA molecule are introduced into a cell, the molecules can be introduced simultaneously or sequentially. For example, targeting endonuclease molecules, each specific for a targeted cleavage site (and optional donor DNA molecules) can be introduced at the same time. Alternatively, each targeting endonuclease molecule, as well as the optional donor DNA molecules can be introduced sequentially.

(c) Culturing the Cell

The method further comprises maintaining the cell under appropriate conditions such that the targeting endonuclease, which is expressed if necessary, binds to and cleaves the targeted chromosomal sequence. The double-stranded break in the chromosomal sequence can be repaired by (i) a non-homologous end-joining repair process such that the chromosomal sequence is modified by a deletion, insertion and/or substitution of at least one nucleotide or (ii) a homology-directed repair process such that the donor sequence of the donor DNA molecule can be integrated into or exchanges with the targeted chromosomal sequence such that the chromosomal sequence is modified.

In general, the cell is maintained under conditions appropriate for cell growth and/or maintenance. Suitable cell culture conditions are well known in the art and are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Cells deficient in cytosolic DNA sensing have (i) higher survival rates after transfection with at least one double-stranded DNA molecule, and (ii) higher rates of targeted genome editing or targeted transgene integration than parental cells not deficient in cytosolic DNA sensing, as detailed above in section (I)(e).

### (III) Compositions

Also provided herein are compositions comprising cells deficient in cytosolic DNA sensing. For example, a composition can comprise a cell deficient in cytosolic DNA sensing and at least one double-stranded exogenous DNA molecule, wherein the cell deficient in cytosolic DNA sensing has a higher survival rate after transfection with the at least one double-stranded exogenous DNA molecule than a parental cell not deficient in cytosolic DNA sensing. Cells deficient in cytosolic DNA sensing are detailed above in section (I). The composition can further comprise cell culture medium or stabilization solution.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, “deficient” refers to reduced or non-detectable levels of the targeted proteins, or reduced or non-detectable activity of the targeted proteins.

As used herein, the term “endogenous sequence” refers to a chromosomal sequence that is native to the cell.

The term “exogenous sequence” refers to a chromosomal sequence that is not native to the cell, or a chromosomal sequence that is moved to a different chromosomal location.

As “engineered” or “genetically modified” cell refers to a cell in which gene expression and/or genomic structure has been modified. For example, the cell can be engineered to comprise an interference agent that represses or disrupts expression of a protein encoded in the genome. Alternatively, the cell can be engineered to have a modified genome, i.e., the cell contains at least chromosomal sequence that has been engineered to contain an insertion of at least one nucleotide, a deletion of at least one nucleotide, and/or a substitution of at least one nucleotide.

The terms “genome modification” and “genome editing” refer to processes by which a specific chromosomal sequence is changed such that the chromosomal sequence is modified. The chromosomal sequence may be modified to comprise an insertion of at least one nucleotide, a deletion of at least one nucleotide, and/or a substitution of at least one nucleotide. The modified chromosomal sequence is inactivated such that no product is made. Alternatively, the chromosomal sequence can be modified such that an altered product is made.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms can encompass known analogs of natural nucleotides, as well as nucleotides that are modified in the base, sugar and/or phosphate moieties. In general, an analog of a particular nucleotide has the same base-pairing specificity; i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site” or “target sequence” refer to a nucleic acid sequence that defines a portion of a chromosomal sequence to be modified or edited and to which a targeting endonuclease is engineered to recognize and bind, provided sufficient conditions for binding exist.

The terms “upstream” and “downstream” refer to locations in a nucleic acid sequence relative to a fixed position. Upstream refers to the region that is 5′ (i.e., near the 5′ end of the strand) to the position and downstream refers to the region that is 3′ (i.e., near the 3′ end of the strand) to the position.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the invention.

### Example 1

### Nucleofection of THP-1 KO (MB21D1) Cells

THP-1 wild type (wt) or THP-1 KO (MB21D1) were nucleofected with Amaxa solution V (comprises a GFP expression vector) or solution SG containing from 1-5 μg of DNA. The cells were incubated at 30° C. for 24 hrs and at 37° C. for 24 hrs. Cell viability was determined using a live/dead cell fluorescent dye (i.e., DRAQ7) and transfection was monitored by GFP fluorescence. The results are present below in Table 1.

TABLE 1
Comparison of THP-1 KO (MB21D1) and THP-1 wt cells.

| | KO (MD21D1) | | Wild type | |
|---|---|---|---|---|
| | Viability | Transfection | Viability | Transfection |
| | (%) | (%) | (%) | (%) |
| Not | 92.1 | 0.3 | 89.4 | 5.3 |
| nucleofected | | | | |
| 1 μg DNA (V) | 68.3 | 51.8 | 27.8 | 46.8 |
| 2 μg DNA (V) | 63.8 | 66.2 | 20.9 | 62.0 |
| 2 μg DNA (SG) | 67.4 | 59.2 | 37.2 | 43.8 |
| 3 μg DNA (V) | 56.* | 76.0 | 16.8 | 59.3 |
| 5 μg DNA (V) | 53.0 | 83.7 | 8.3 | 63.4 |

Wild type THP-1 cells nucleofected with 1 μg of DNA had a viability of 28%, whereas THP-1 cells without a functional MB21D1 had a viability of 68% under the same nucleofection conditions. Nucleofection 5 μg of DNA killed 92% of THP-1 wt cells but only 47% of the KO (MB21D1) THP-1 cells.

### Example 2

### Nucleofection of Other Hematopoietic Cell Lines

Similar transfections were performed in three other hematopoietic cell lines (HL-60, U-937, and Ramos). The cells were subjected to 24 hrs of cold shock followed by 24 hrs at 37° C. Cell viability was determined using a live/dead cell fluorescent dye (i.e., DRAQ7) and transfection was monitored by GFP fluorescence. The results are shown in Table 2.

TABLE 2
Transfection in hematopoietic cell lines.

| | HL-60 | | U-937 | | Ramos | |
|---|---|---|---|---|---|---|
| | % | % | % | % | % | % |
| | viability | transfct | viability | transfct | viability | transfct |
| No transf | 88.0 | 0.3 | 73.0 | 0.1 | | |
| 1 μg DNA | 40.0 | 40.0 | 75.0 | 22.0 | | |
| 2 μg DNA | 28.0 | 44.0 | 70.0 | 40.0 | 47.0 | 6.0 |
| 2 μg DNA | 33.0 | 64.0 | | | | |
| 2 μg DNA | 46.0 | 54.0 | | | | |
| 3 μg DNA | | | | | 34.0 | 10.0 |
| 4 μg DNA | | | | | 31.0 | 11.0 |
| 5 μg DNA | 20.0 | 58.0 | 40.0 | 48.0 | 45.0 | 8.0 |
| 6 μg DNA | | | | | 36.0 | 17.0 |

### Example 3

### Targeted Integration into the Actin Locus of THP-1 KO (MB21D1) Cells

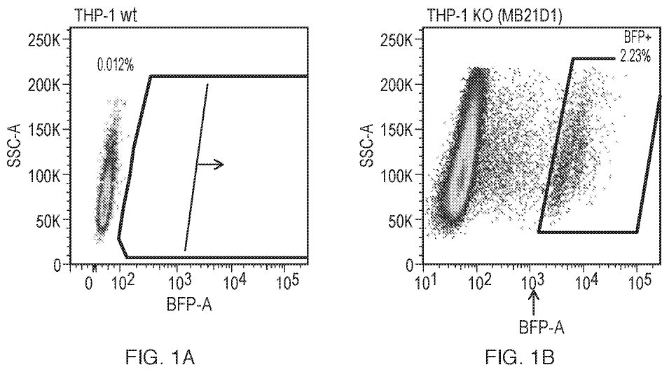

Using ZFNs targeted to the actin locus and a donor plasm id (2 μg) comprising a fluorescent protein flanked by homolgous sequences, targeted integration of the fluorescent protein can occur within the actin locus of wild type THP-1 cells. However, the rate of integration was so extremely low rate (about 0.012%) that multiple enrichments were needed to isolate the integrants. THP-1 KO (MB21D1) cells were nuclefected with actin ZFNs and a donor plasmid (5 μg) comprising blue flourescent protein (BFP). The rate of targeted integratrion into the actin locus drastically increased to 2.23% in the MB21D1 knock-out cells. See FIG. 1.

### Example 4

### Targeted Integration into the Tubulin Locus of THP-1 KO (MB21D1) Cells

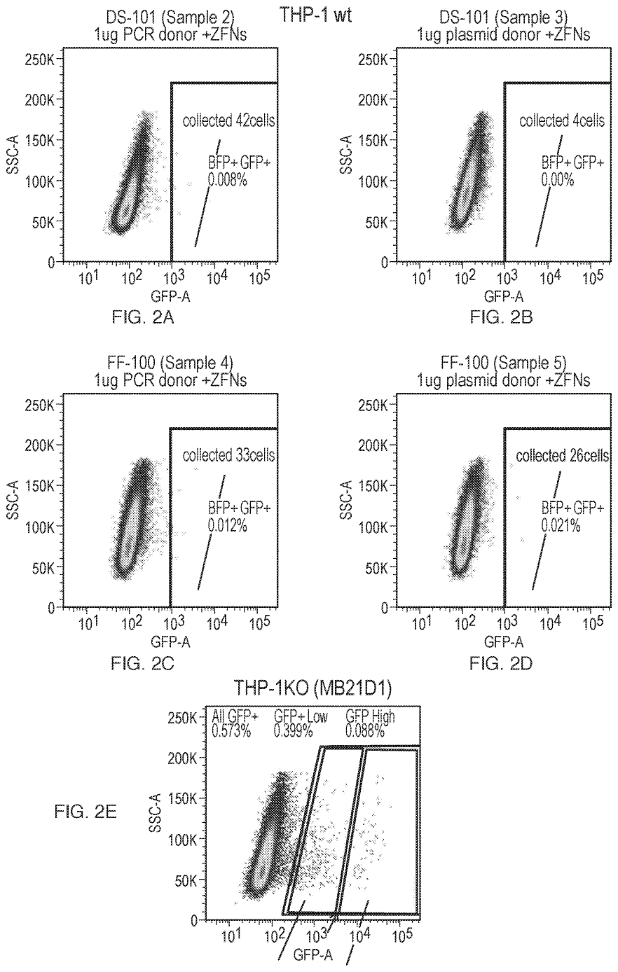

Despite repeated attempts, targeted integration into the tubulin locus of THP-1 cells was never successful. Using THP-1 KO (MB21D1) cells, however, integration of GFP in the tubulin locus occurred at a high rate on the first attempt. See FIG. 2.

1. A method for increasing targeted genome editing, the method comprising introducing a targeting endonuclease or a nucleic acid encoding the targeting endonuclease and optionally a donor DNA molecule into a cell deficient cytosolic DNA sensing, wherein the cell deficient in cytosolic DNA sensing has a higher rate of targeted genome editing than its parental cell not deficient in cytosolic DNA sensing.

2. The method of claim 1, wherein the cell deficient in cytosolic DNA sensing is engineered to lack or have a reduced level of a protein involved in cytosolic DNA sensing.

3. The method of claim 2, wherein the protein involved in cytosolic DNA sensing is chosen from cyclic GMP-AMP synthase (cGAS), stimulator of interferon genes (STING), interferon gamma inducible protein 16 (IFI16), DEAD-box helicase 41 (DDX41), leucine rich repeat (in flightless I) interacting protein (LRRFIP1), or combinations thereof.

4. The method of claim 2, wherein the cell deficient in cytosolic DNA sensing comprises at least one inactivated chromosomal sequence encoding the protein involved in cytosolic DNA sensing.

5. The method of claim 4, wherein the inactivated chromosomal sequence was inactivated with a targeting endonuclease-mediated genome editing technique.

6. The method of claim 2, wherein the cell deficient in cytosolic DNAsensing comprises an RNA interference (RNAi) or a CRISPR interference (CRISPRi) agent.

7. The method of claim 5, wherein the targeting endonuclease is chosen from a zinc finger nuclease, a clustered regularly interspersed short palindromic repeats (CRISPR)/CRISPR-associated (Cas) (CRISPR/Cas) nuclease system, a CRISPR/Cas dual nickase system, a transcription activator-like effector nuclease, a meganuclease, or a fusion protein comprising a programmable DNA-binding domain and a nuclease domain.

8. The method of claim 7, wherein the nucleic acid encoding the targeting endonuclease is mRNA or DNA.

9. The method of claim 1, wherein the donor DNA molecule comprises a donor sequence that is flanked by at least one sequence having substantial sequence identity with a sequence at or near a genomic site that is targeted by the targeting endonuclease.

10. The method of claim 1, wherein the cell deficient in cytosolic DNA sensing is a mammalian cell.

11. The method of claim 10, wherein the mammalian cell is a hematopoietic cell line.

12. The method of claim 11, wherein the hematopoietic cell line is chosen from THP-1, HL-60, U-937, Ramos, or Jurkat.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A composition comprising a cell deficient in cytosolic DNA sensing and at least one double-stranded exogenous DNA molecule, wherein the cell deficient in cytosolic DNA sensing has a higher survival rate after transfection with the at least one double-stranded exogenous DNA molecule than a parental cell not deficient in cytosolic DNA sensing.

27. The composition of claim 26, wherein the cell deficient in cytosolic DNA sensing is engineered to lack or have a reduced level of a protein involved in cytosolic DNA sensing.

28. The composition of claim 27, wherein the protein involved in cytosolic DNA sensing is chosen from cyclic GMP-AMP synthase (cGAS), stimulator of interferon genes (STING), interferon gamma inducible protein 16 (IFI16), DEAD-box helicase 41 (DDX41), leucine rich repeat (in flightless I) interacting protein (LRRFIP1), or combinations thereof.

29. The composition of claim 27, wherein the cell deficient in cytosolic DNA sensing comprises at least one inactivated chromosomal sequence encoding the protein involved in cytosolic DNA sensing.

30. The composition of claim 29, wherein the inactivated chromosomal sequence was inactivated with a targeting endonuclease.

31. The composition of claim 27, wherein the cell deficient in cytosolic DNA sensing comprises an RNA interference (RNAi) or a CRISPR interference (CRISPRi) agent.

32. The composition of claim 26, wherein the at least one double-stranded exogenous DNA molecule encodes a targeting endonuclease and/or comprises a donor sequence for targeted integration.

33. The composition of claim 26, wherein the cell deficient in cytosolic DNA sensing is a mammalian cell.

34. The composition of claim 33, wherein the mammalian cell is a hematopoietic cell line.
